(19) United States
(12) Patent Application Publication
(10) Pub. No.: US 2006/0167527 A1
(43) Pub. Date: Jul. 27, 2006

(54) Apparatus and method for repair of spinal cord injury

(76) Inventors: Philip A. Femano, Nutley, NJ (US); Michael J. Zanakis, Palm City, FL (US)

Correspondence Address: Mintz, Levin, Cohn, Ferris,;Glovsky and Popeo, P.C., The Chrysler Center, 666 Third Avenue, 24th Floor, New York, NY, 10017, US

(21) Appl. No.: 11/301,735

(22) Filed: Dec. 12, 2005

Related U.S. Application Data
(63) Continuation-in-part of application No. 10/292,414, filed on Nov. 11, 2002, now Pat. No. 6,975,907.
(60) Provisional application No. 60/350,490, filed on Nov. 13, 2001.

(51) Int. Cl. A61N 1/18

(52) U.S. Cl. 607/50

(57) ABSTRACT

An apparatus for stimulating regeneration and repair of damaged spinal nerves, comprising at least two electrodes placed intravertebrally near the site of spinal neurite injury and delivering direct current thereto. A method for stimulating regeneration and repair of damaged spinal nervous tissue, comprising placing electrodes intravertebrally near the site of spinal cord injury and applying direct current at a level sufficient to induce regeneration and repair of damaged spinal neurites but less than the current level at which tissue toxicity occurs.

### CROSS-REFERENCE TO RELATED PATENT APPLICATIONS

This application is a continuation-in-part of U.S. patent application Ser. No. 10/292,414 filed Nov. 11, 2002, which claims priority from U.S. Provisional Patent Application Ser. No. 60/350,490. filed Nov. 13, 2001. All patents, patent applications, and references cited in this specification are incorporated by reference in their entirety.

### BACKGROUND OF THE INVENTION

1. Field of the Invention

The present invention relates to an apparatus and method for repairing spinal cord injury, and specifically an apparatus and method for stimulating regeneration and repair of damaged spinal nervous tissue.

2. Discussion of the Related Art

Spinal cord injury occurs when the normal function of axons or other neural fibers of the spinal cord (collectively: neurites) is interrupted, generally by mechanical forces. If the spinal cord is compressed, severed or contused, the physical or physiological integrity of neurites may be compromised, so that insufficient conduction of neuroelectric impulses can occur along the affected neurite's length. Eventually, large populations of neurites, including their associated cell bodies, may die, causing massive loss in communication between the brain and the peripheral nerves, and resulting in varying degrees of paraplegia or quadriplegia.

Studies show that spinal cord injuries may be repaired if damaged spinal neurites can be induced to regenerate. Such regeneration and repair can be induced by ultra-low level electric field stimulation, provided that the electric field is produced by direct current (DC). The DC field is far below the electrical threshold for generating action potentials or any other known functional electrical activity in neurites and serves to promote a regenerative phenomenon that appears to be initiated by a substantial number of neurites, and also serves to guide neurites toward the cathode of the electric field. As neurites appear to respond to the field strength of exogenously applied fields, as opposed to the total current or voltage applied, neurite growth and directional guidance are the key effects of DC electric field application.

Neurite growth and directional guidance are not well understood. It is thought that there may be an optimum electric field strength for regeneration and repair, while directionality is a function of the flux density, electric gradient, and the orientation of the flux lines produced by the electric field. Unfortunately, the density at which unbalanced direct current can be applied to nervous tissue is finite, with the upper limit being the level of toxicity where significant cell damage occurs. The maximum safe current is approximately 75 micro-amps per square centimeter of the surface area of the conductive electrode interfacing with the tissue.

Existing electrode designs have attempted to minimize localized toxic effects of current application to the spinal cord by using extravertebral electrodes. However, extravertebral electrodes require significant amounts of power to produce effective field strengths within the damaged spinal cord. This is because extravertebral placement of the electrodes means that the anode and cathode are physically remote from the site of injury. As a result, more power is required to generate the requisite electric field to the injury site, potentially resulting in toxic effects to tissues in the immediate vicinity of the conductive electrode surface, such as muscle, nerves and blood vessels. It is understood that regeneration and repair of spinal neurites is counterproductive if the muscles to be controlled or their associated blood vessels and nerves are damaged as a result.

Further, extravertebral placement of electrodes can result in situating the electrodes lateral to the site of the spinal cord injury, rather than in line therewith, resulting in less than optimal directional neurite guidance by the cathodal current. Still further, extravertebral placement of electrodes affects the extent to which the electrical flux lines generated by the electrodes deviate from the ideal, which itself is a major determinant in the quality of the electrical field established in the spinal cord. When electrodes are situated in extravertebral muscle, the flux lines within the spinal cord can be distorted from ideal by each intervening tissue that has a resistivity/conductivity differing from that of the muscle. The tissues that vary in these parameters and through which the current must pass, in the case of extravertebral placement of electrodes, include bone, ligaments, fat, cerebrospinal fluid, and vasculature. These structures may act as additional resistance or current shunts that can serve to deviate the resulting electric field within the spinal cord from a nominal field. Extravertebral field application is rendered significantly less reliable and thus less efficacious as the result of the difficulty in predicting the effects of the different resistivity/conductivity parameters of the intervening tissues.

What is needed is an apparatus and method for stimulating regeneration and repair of damaged spinal neurites whereby control over the local electric field within the spinal cord is optimized, and toxicity to the central nervous system (CNS) and other tissues is minimized.

Accordingly, the present invention provides an apparatus suited to intravertebral implantation at the site of spinal cord injury, that allows DC stimulation of the injury site sufficient to induce regeneration and repair of damaged neurites, but at a current below the nontoxic threshold of 75 micro-amps per square centimeter.

The present invention also provides a method for stimulating regeneration and repair of damaged spinal neurites through intravertebral implantation of electrodes at the site of spinal cord injury, and DC stimulation at the injury site sufficient to induce regeneration and repair of the damaged neurites, but at a current level below the level at which tissue toxicity occurs.

### BRIEF SUMMARY OF THE INVENTION

An aspect of the present invention includes at least two electrodes configured to be placed intravertebrally proximal to the site of spinal neurite injury and deliver direct current (DC) thereto. Each electrode includes an aggregate conductive electrode surface sufficiently large such that the current density from the electrode surface will induce neurite regeneration and repair without damaging the surrounding tissue. In a preferred embodiment, the aggregate electrode surface includes multiple conductive sub-surfaces. The conductive sub-surfaces are separated from each other by non-conducting septa to minimize the production of, and dissipate, any toxic product, such as free ionic protons, developed as the result of the delivery of electric current.

Another aspect of the present invention includes placing the electrodes of the present invention intravertebrally proximal to the site of spinal cord injury and applying direct current at a level sufficient to induce regeneration and repair of damaged spinal neurites but less than the current level at which tissue toxicity occurs. The current is applied for a duration sufficient to prevent significant die-back and achieve net growth.

In a preferred embodiment, the electrodes are arrayed so as to encompass a cross-sectional area of the spinal cord, in the area of the spinal neurite injury. In another preferred embodiment, the electrodes are arrayed in a three-dimensional geometry, such as a triangle, surrounding the site of spinal neurite injury.

In one aspect of the present invention, the direct current is applied for sufficient duration to prevent significant die-back, ensuring that forward-direction neurite regeneration and repair prevails over die-back.

### BRIEF DESCRIPTION OF THE DRAWINGS

FIG. 1 depicts three preferred configurations for the aggregate conductive electrode surface of the apparatus of the present invention.

FIG. 2 is a graph of the electrode current profile across a single conductive electrode surface as a function of the relative distance across a single conductive electrode surface for each configuration depicted in FIG. 1.

FIG. 3 depicts the electrode surface of the apparatus of the present invention, showing various patterns of separation between adjacent conductive sub-surfaces on the conductive electrode surface.

FIG. 4 is a graph of the relationship of toxic product concentration in the tissue as a function of the separation between adjacent conductive sub-surfaces on the conductive electrode surface.

FIG. 5 is a schematic representation of one embodiment of the invention. 1, 2, 3, 7, 8 and 9 represents intravertebral electrodes intravertebrally implanted in a triangle arrangement. 4, 5 and 6 are three segments of the spinal cord where 5 is a segment comprising an injury. It is understood that the invention is not limited to FIG. 5, which is merely one embodiment of the invention. Many other embodiments are envisioned and described throughout this disclosure.

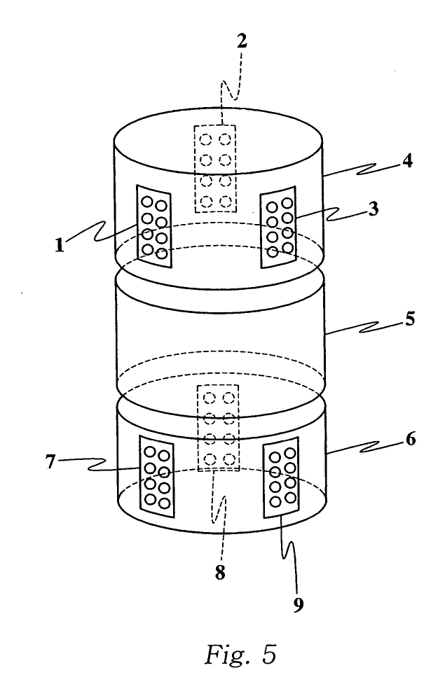

### DETAILED DESCRIPTION OF THE INVENTION

The apparatus for stimulating regeneration and repair of damaged spinal neurons of the present invention includes at least two electrodes that are configured to be placed intravertebrally proximal the site of spinal neurite injury and deliver direct current thereto. The electrodes include an aggregate conductive electrode surface through which the direct current is delivered to the injury site. The aggregate conductive electrode surface is sufficiently large so that the density of the delivered direct current can induce neurite regeneration and repair without generating a significant amount of toxic product in surrounding tissues.

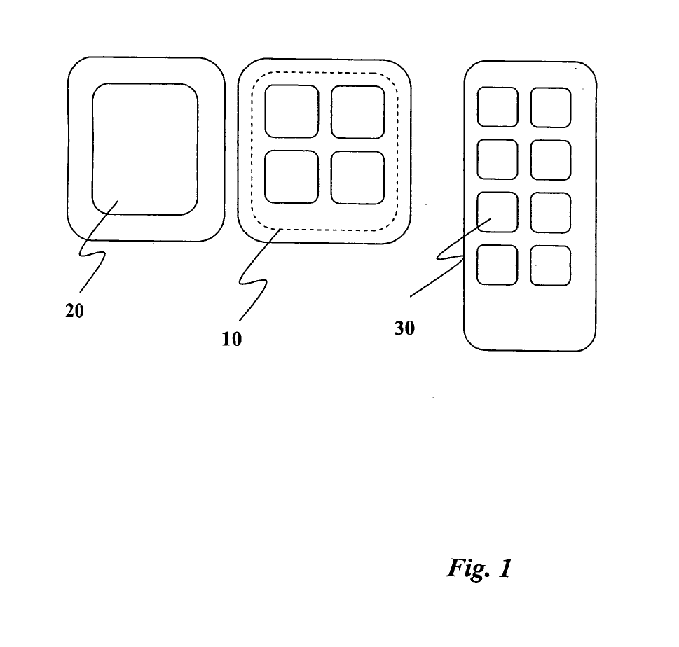

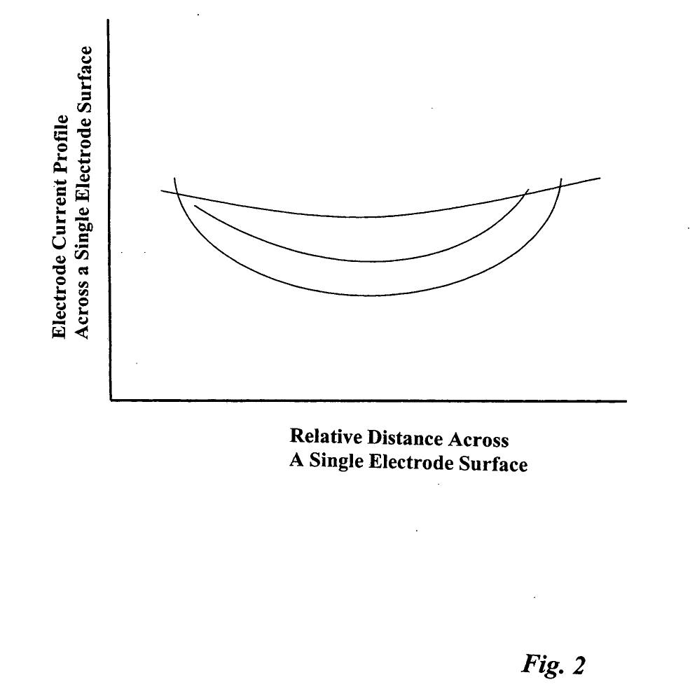

As shown in FIG. 1, the aggregate conductive electrode surface 10 may include a single conductive surface 20 or multiple conductive sub-surfaces 30. Where multiple conductive sub-surfaces 30 are used, the result is a flattening of the trans-surface current gradient, or “skin effect,” across each sub-surface. As shown in FIG. 2, the benefit is that regeneratively efficacious currents can be delivered to the injury site while minimizing the delivery of toxic peak currents. In FIG. 2, the uppermost curve shows the “skin effect” for multiple conductive sub-surfaces. The middle curve shows the “skin effect” for a smaller number of conductive sub-surfaces, and the lowermost curve shows the “skin effect” for a single conductive sub-surface.

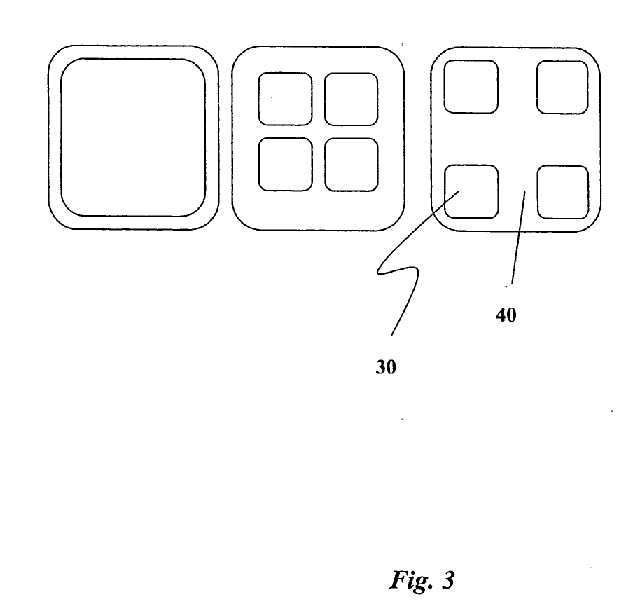

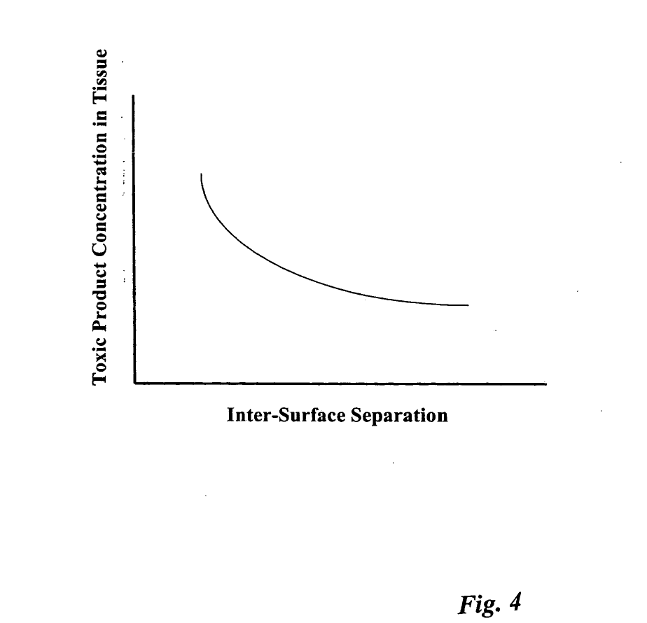

Further where the aggregate electrode surface includes multiple conductive sub-surfaces 30, adjacent sub-surfaces 30 are separated by non-conducting septa 40, as shown in FIG. 3. The left figure shows no septum between conductive surfaces. The center figure shows a small septum between the adjacent conductive surfaces. The right figure shows a large nonconductive septum between adjacent conductive subsurfaces. The specific geometry of the non-conducting septa 40 relative to the conductive sub-surfaces 30 may vary as required to optimize the contribution of the septal effect. Specifically, interposing non-conducting septa 40 between adjacent sub-surfaces 30 reduces the concentration, in surrounding tissues, of any toxic product developing as a result of the delivery of electric current through the electrode, by virtue of the dissipation of the toxic product across the total area of the entire aggregate conductive electrode surface 10. The aggregate electrode surface includes conductive surfaces, either a single conductive surface 20 or multiple conductive sub-surfaces 30, and non-conductive septa 40. FIG. 4 shows the relationship between the dilution of toxic product and the size of the non-conductive septa. The non-conducting septa 40 may constitute empty space between adjacent conductive sub-surfaces 30.

The apparatus of the present invention may also be arrayed for use in neural systems having multi-directional neurite elements. In such systems, the electrical field may be applied sequentially along the direction of each damaged neurite population. Accordingly, the location of stimulating electrodes can vary depending on the direction along which regeneration and repair is sought, so that discrete intravertebral multi-electrode surfaces can be used to stimulate neurite growth in a selective fashion. For example, it is known that dorsally-situated neurites will regenerate rostrally, while corticospinal neurites, situated laterally, will regenerate caudally. Thus, an intravertebral panel comprising a plurality of electrodes encompassing the cross-sectional area of the spinal cord can selectively and safely produce cathodally-directed current for longer periods of time over the neurite tracts of interest.

Alternatively, electrodes may be configured in a three-dimensional geometry, such that the aggregate electrode stimulation through multiple electrodes can generate an effective electrical field along any desired vector.

The number of electrodes in a given paradigm, the specific geometric placement of the electrodes, and the aggregate use of a plurality of electrodes may vary according to the demands of the therapeutic challenge for which DC stimulation is being applied.

According to the method of the present invention, the electrodes as described are placed intravertebrally proximal to the site of spinal injury. Once the electrodes are so placed and properly arrayed, direct current is delivered through the electrodes to the injury site, inducing regeneration and repair of spinal neurites. The current density of the delivered direct current is sufficient to induce neurite regeneration and repair while avoiding tissue toxicity. Preferably, the current density at the electrode-tissue interface is less than 75 micro-amps per square centimeter. As relatively high resistivity tissues such as bone and fat are located distal to the desired locus of electrical field regeneration and repair, in intravertebral stimulation the bone, fat and meninges serve as a natural physical guidance means to provide a directional path for neurite regeneration and repair. In this way, intravertebral regeneration and repair may represent an improvement over nerve regeneration and repair systems in which a physical guidance system is actively employed. At the same time, intravertebral electrode placement allows the safe delivery of higher currents to the injury site, so that higher field strengths can be injected thereto. Since the electrodes are applied locally, the relative amount of current delivered can be low, relative to extravertebral electrodes, and yet may achieve field strengths higher than extravertebral electrodes can achieve.

The duration of electrical stimulation is sufficient to prevent significant “die-back” phenomenon, as explained by McCaig, in “Spinal Neurite Reabsorption and Regrowth in vitro Depend on the Polarity of an Applied Electric Field,” Development 100, 31-41 (1987), and which is incorporated herein by reference. The optimal stimulation duration will depend upon the specific therapeutic application. The duration will be sufficient to ensure that the forward-direction regenerative neurite growth prevails over the “die-back” effect.

DC stimulation of damaged spinal neurites may be used as a stand-alone regenerative and repair therapy, or may be used as an adjunct to other therapies, whether presently available or to become available in the future. Such therapies include, but are not limited to, pharmaceutical, genetically-engineered, biological, surgical, psycho- and physical therapies.

The electrode, including the electrode surface, may be made from conventional materials. The direct current may be generated from any conventional DC generator used in biotherapeutic applications.

By way of example, a number of specific embodiments of the invention are disclosed below. It is understood that these specific embodiments, in any combination, may be used in any of the methods, apparatus, and electrodes of the invention.

One embodiment of the invention is directed to an apparatus for stimulating regeneration and repair of damaged spinal nervous tissue, comprising a plurality of electrodes configured to be placed intravertebrally proximal to a site of spinal neurite injury and deliver direct current from a DC source thereto, each of said plurality of electrodes including an aggregate conductive electrode surface through which said direct current is delivered, said aggregate conductive electrode surface being sufficiently large so that a current density from said electrode surface will induce neurite regeneration and repair while minimizing damage to tissue surrounding the site of spinal neurite injury. To minimize damage to tissue surrounding the site of spinal neurite injury, it is preferred to limit the apparatus to less than 150 micro-amps of electricity per square centimeter of conductive area of one polarity. In a preferred embodiment, this apparatus is limited to less than 120 micro-amps, less than 100 micro-amps, less than 90 micro-amps, or less than 75 micro-amps of electricity per square centimeter of conductive area of one polarity. In other words, an apparatus with a one square centimeter of positive conductive electrode surface and one square centimeter of negative conductive electrode surface should have its current limited to less than 150 micro-amps, less than 120 micro-amps, less than 100 micro-amps, less than 90 micro-amps, or less than 75 micro-amps. It is understood that the conductive area of one polarity may be split between a plurality of conductive sub-surfaces in each electrode. Since the methods and apparatus may comprise multiple electrodes, it is understood that the conductive area of one polarity may be split among multiple electrodes. For example, the methods and apparatus of the invention may use six electroded (each with a plurality of conductive subsurfaces) where 3 electrodes are positive and 3 electrodes are negative.

The harmful effects of prolonged contact with a direct current can be reduced by manufacturing the aggregate conductive electrode surface with a plurality of conductive sub-surfaces where the subsurface is surrounded by non-conductive septa (See, e.g., FIGS. 1 and 3). In other words, the conductive subsurface is separated from each other by non conductive septa. As toxic chemical byproducts are produced during prolonged direct current application, these non-conductive septa regions allow the such byproducts to diffuse away from the conductive area to be dissipated or neutralized by the body's natural functions. Thus, the septa serving as a safety measure, allows an otherwise toxic application of electrical stimulation to become less toxic or nontoxic.

In a preferred embodiment, each electrode, including the aggregate conductive electrode surface and the conductive sub-surfaces of the electrode, are all electrically connected and deliver current on one polarity at one time only. That is, the one or more conductive sub-surfaces of one electrode is either all positive in polarity or all negative in polarity. The polarity or magnitude may change or reverse in time but any two conductive sub-surfaces of one electrode should never have opposite polarity.

Another embodiment of the invention is directed to a novel electrode (referred to below as the “electrode”) which may be used in any of the apparatus or methods of the invention. The electrode comprises an aggregate conductive electrode surface. The aggregate conductive electrode surface, in turn, comprises a plurality of conductive sub-surfaces which are separated from each other by non-conducting septa. The septa is of sufficient size (total area) to minimize production of and dissipate toxic products generated from a delivery of direct current intravertebrally proximal to a site of spinal cord injury. Further, each of the conductive sub-surfaces are connected to each other by at least one electrical connection.

The septa (or non-conductive surface) of the aggregate conductive electrode surface may be at least a percentage of the total aggregate conductive electrode surface area. In a preferred embodiment, the percentage is 1%. In more preferred embodiments, this percentage may be 10%, 20%, 30%, 40%, 50%, 60% or 70%.

Electrical connections for connecting conductive sub-surfaces are known in the art. For example, the aggregate conductive electrode surface may comprise a subsurface layer which comprises tracings which electrically connect each sub-surface. In a preferred embodiment, the electrical connection is made by non-corrosive connecting wires. Methods for connecting “connecting wires” to conductive surfaces are known in the art and include, at least the use of soldering connections, crimping connections and specially designed connectors. In a preferred embodiment, the connecting wires are connected to the approximate center area of a conductive sub-surface. In another preferred embodiment, each conductive subsurface is connected to a plurality of connecting wires. For example, a conductive sub-surface may be connected to 1, 2, 3, 5 or 10 connecting wires. The connections may be, for example, evenly or roughly evenly distributed across the electrically insulated region of the conductive subsurface. Furthermore, the electrical connections may be made in series, in parallel or a combination of both. For example, in an aggregate conductive electrode surface with three subsurfaces A, B and C, all three sub-surfaces may be connected to a single terminal (for connection to a DC source) in a parallel configuration. Alternatively, A may be connected to the terminal, B may be connected to A, and C may be connected to B in a series connection. Further, A and B may be connected to the terminal and C may be connected to B in a mixed series and parallel configuration. Also, A, B and C may each be connected to a terminal and each may be connected to each other in a mixed series and parallel configuration.

Since the electrode is designed for the practice of the methods of the invention, it is designed with dimensions for easy intravertebral placement. For example, the electrode may have an aggregate conductive electrode surface with a length, along any dimension of less than 50 mm, less than 45 mm, less than 40 mm, less than 35 mm, less than 30 mm or less than 25 mm.

The conductive sub-surfaces may be of any geometric shape including random shapes. In a preferred embodiment, the subsurfaces may be oval, circular, square, rectangular, polygon and the like. Further, where the shape is a polygon or where the shape contains sharp corners, the corners may be rounded such as, for example, a square with round corners, triangle with rounded corners and rectangular with round corners. Naturally, each aggregate conductive electrode surface may have conductive sub-surfaces of the same shape, or of different shapes, or a mixture of same and different shapes together. In a preferred embodiment, the conductive sub-surfaces are designed to minimize the circumference to area ratio. In this embodiment, a round sub-surface is preferred although any shape that is identical, or roughly identical, in length and width is also preferred. Such shapes include circles, squares, rectangles, triangles, pentagons, hexagons and octagons where the height to width ratio (height/width) is between 2 to 0.5.

The conductive sub-surfaces of an aggregate conductive electrode surface may be arranged in any fashion including one or more columns (i.e., 1, 2, 3, 4, 5, 10 or more columns etc) or in a random or other geometric (e.g., in a circle) arrangement. The column configuration may be of any arrangements or combination of arrangements including parallel columns, radially separated columns (e.g., complete or partial spoke on a wheel pattern), intersecting column (e.g., in the shape of a cross, an x y grid), randomly spaced columns, randomly spaced and intersecting column etc.

In a preferred embodiment, the electrode comprises a single terminal only. The single terminal may be used for connecting the electrode directly or indirectly to a power source. The single terminal may connect to the power source directly or the connection may be made indirectly through leads, wires, extensions, and the like. In this embodiment, applying electric current through the terminal will cause all the sub-surfaces of the electrode to have the same polarity and to have the same current direction.

All the subsurfaces, leads, terminals, and electrical connections of the invention may be made completely, or in part, of a material which is not corrosive when conducting direct current in a intravertebral location. Examples of such materials include platinum, iridium, carbon and alloys (where applicable) and combinations of these materials. Combinations refers to a device made of two materials such as a platinum wire with iridium ends and the like.

While the invention has been described with respect to certain specific embodiments, it will be appreciated that many modifications and changes may be made by those skilled in the art without departing from the invention. It is intended, therefore, by the appended claims to cover all such modifications and changes as may fall within the true spirit and scope of the invention.

In this disclosure, it is understood that the term neurites also encompasses axons. It follows that wherever the words neurite or neurites are used, it can be substituted with the words axon or axons respectively.

1. An apparatus for stimulating regeneration and repair of damaged spinal nervous tissue, comprising a plurality of electrodes configured to be placed intravertebrally proximal to a site of spinal neurite injury and deliver direct current from a DC source thereto, each of said plurality of electrodes including an aggregate conductive electrode surface through which said direct current is delivered, said aggregate conductive electrode surface being sufficiently large so that a current density from said electrode surface will induce neurite regeneration and repair while minimizing damage to tissue surrounding the site of spinal neurite injury.

2. The apparatus of claim 1, wherein said aggregate conductive electrode surface is comprised of a plurality of conductive sub-surfaces, said conductive sub-surfaces being separated from each other by non-conducting septa sufficient to minimize production of and dissipate toxic product generated from said delivery of direct current to said site of spinal neurite injury.

3. The apparatus of claim 2, wherein each of said plurality of conductive sub-surfaces on one electrode delivers a direct current of one polarity at a time only.

4. The apparatus of claim 1 wherein said direct current is delivered at said conductive sub-surface at a current density of less than 150 micro-amps per square centimeter of conductive sub-surface area.

5. The apparatus of claim 1 wherein said direct current is delivered at said conductive sub-surface at a rate of less than 75 micro-amps per square centimeter of conductive sub-surface area.

6. An electrode comprising an aggregate conductive electrode surface, said aggregate conductive electrode surface comprising a plurality of conductive sub-surfaces which are separated from each other by non-conducting septa sufficient to minimize production of and dissipate toxic products generated from a delivery of direct current intravertebrally proximal to a site of spinal cord injury, wherein each of said conductive sub-surfaces are connected to each other by at least one electrical connection.

7. The electrode of claim 6 wherein said electrical connection comprises at least one non-corrosive connecting wires.

8. The electrode of claim 6 wherein each said conductive sub-surface is connected in its center to at least one of said at least one connecting wires.

9. The electrode of claim 6 wherein said plurality of conductive sub-surfaces are connected in parallel to a common terminal.

10. The electrode of claim 6 wherein said plurality of conductive sub-surfaces are connected in series.

11. The electrode of claim 6 wherein said conductive sub-surfaces have a shape selected from the group consisting of oval, circular, square, rectangular, square with round corners and rectangular with round corners.

12. The electrode of claim 6 wherein each said conductive sub-surfaces is substantially identical in length and width.

13. The electrode of claim 6, wherein said plurality of conductive sub-surfaces are arranged in one column.

14. The electrode of claim 6, wherein said plurality of conductive sub-surfaces are arranged in two columns.

15. The electrode of claim 6, wherein said plurality of conductive sub-surfaces are arranged in a random order.

16. The electrode of claim 6, wherein said electrode further comprises a single terminal for connecting to a direct current power source, wherein said terminal is electrically connected to all the conductive sub-surfaces of said electrode.

17. The electrode of claim 6 wherein said conductive sub-surfaces is made of material which is not corrosive when conducting direct current in an intravertebral location.

18. The electrode of claim 17, wherein said non-corrosive material is selected from the group consisting of platinum, iridium, carbon and alloys and combinations thereof.

19. The electrode of claim 6 which has a length along any one dimension of less than or equal to 50 mm.

20. The electrode of claim 6 which has a length along any one dimension of less than or equal to 30 mm.

21. The electrode of claim 6 wherein said nonconductive septa comprise an area of at least 1% of the surface said aggregate conductive electrode surface.

22. The electrode of claim 6 wherein said nonconductive septa comprise an area of at least 30% of the surface said aggregate conductive electrode surface.

23. The electrode of claim 6 wherein said nonconductive septa comprise an area of at least 50% of the surface said aggregate conductive electrode surface.
